(19) United States
(12) Patent Application Publication
(10) Pub. No.: US 2013/0309705 A1
(43) Pub. Date: Nov. 21, 2013

(54) METHOD FOR DETERMINING THE CONCENTRATION OF HMG-COA REDUCTASE INHIBITORS

(71) Applicants: Zhenwen Duan, Beijing (CN); Shuren Guo, Beijing (CN); Xuemei Li, Beijing (CN)

(72) Inventors: Zhenwen Duan, Beijing (CN); Shuren Guo, Beijing (CN); Xuemei Li, Beijing (CN)

(73) Assignee: BEIJING PEKING UNIVERSITY WBL BIOTECH CO. LTD, Beijing (CN)

(21) Appl. No.: 13/983,044

(22) PCT Filed: Jan. 12, 2012
(86) PCT No.: PCT/CN12/70269

§ 371 Date: Aug. 2, 2013

(30) Foreign Application Priority Data
Feb. 1, 2011 (CN) 201110034241.4

(51) Int. Cl. C12Q 1/26

(52) U.S. Cl. CPC C12Q 1/26
USPC 435/25

(57) ABSTRACT

The invention, which belongs to the field of enzymology and pharmaceutical chemistry, relates to a method for determining the concentration of a HMG-CoA reductase inhibitor, and to a method for determining the inhibition rate of HMG-CoA reductase. In particular, the method for determining the inhibition rate of HMG-CoA reductase comprises the following steps: 1) establishing the following enzymatic reaction systems for HMG-CoA reductase: a reaction system for a sample to be tested, to which a sample to be tested is added, and a negative control reaction system, to which deionized water in the same volume as a sample to be tested is added; 2) determining the MVAL concentration in a negative control reaction system and in a reaction system for a sample to be tested by HPLC-MS/MS method, respectively; 3) calculating the inhibition rate of HMG-CoA reductase according to the formula: inhibition rate of HMG-CoA reductase=[(MVAL concentration in a negative control reaction system−MVAL concentration in a reaction system for a sample to be tested)/MVAL concentration in a negative control reaction system]×100%. The inhibition rate of HMG-CoA reductase can be accurately determined by the method.

### TECHNICAL FIELD

The invention, which belongs to the field of enzymology and pharmaceutical chemistry, relates to a method for determining the concentration of a HMG-CoA reductase inhibitor and a method for determining the inhibition rate of HMG-CoA reductase.

### BACKGROUND

HMG-CoA reductase (3-hydroxy 3-methylglutaryl coenzyme A reductase), the rate-limiting enzyme during the synthesis of cholesterol in hepatocytes, catalyzes the production of Mevalonic Acid (MVA). The inhibition of HMG-CoA reductase can restrict the synthesis of cholesterol. Currently, common HMG-CoA reductase inhibitors are statin compounds, e.g. lovastatin hydroxy acid.

Methods for determining the inhibition rate of HMG-CoA reductase include three steps, i.e. enzyme preparation, reaction and determination of inhibition rate. Firstly, a HMG-CoA reductase solution of rat liver microsome was prepared. Furthermore, the prepared enzyme and substrate were placed in a buffer and were reacted with each other for a certain period, and the reaction was terminated by adding acid. After termination of the reaction, the absorbance of the enzymatic reaction system was directly determined by a microplate reader, or the product, such as Mevalonolactone (MVAL), was isolated from the enzymatic reaction system and the content thereof was determined. Currently, methods for determining the content of a product (MVAL) include methods for determining isotope activity by scintillation counter (Kuroda M, Endo A. Inhibition of cholesterol synthesis in vitro by fatty acids. Biochim Biophys Acta, 1976, 486:70; Endo A, et al. Competitive inhibition of 3-hydroxy-3-methylglutaryl coenzyme a reductase by ML-236A and ML-236B fungal metabolites, having hypocholesterolemic activity. FEBS Letters, 1976, 72:323; Alberts A W, et al. Mevinolin: a Highly potent competitive inhibitor of hydroxymethylglutaryl-coenzyme A reductase and a Cholesterol-Lowering Agent. Proc Natl Acad Sci USA, 1980, 77 (7): 3957), LC-MS method (Park E J, et al. Analysis of 3-hydroxy-3-methylglutaryl coenzyme a reductase inhibitors using liquid chromatography-electrospray mass spectrometry. J Chromatogr B Biomed Sci Appl, 2001, 754:327), and LC-ESI-MS/MS method (Honda A, Mizokami Y, Matsuzaki Y, et al. Highly sensitive assay of HMG-CoA reductase activity by LC-ESI-MS/MS. J Lipid Res, 2007, 48:1212).

In the above-mentioned methods, spectrophotometry using a microplate reader has the shortcomings of complex enzyme reaction systems and excessive interference, and thus cannot accurately reflect the absorbance of a certain substrate (NADPH) and can only be applied to qualitative analysis. Among the methods for determining the content of a product (MVAL), the methods for determining isotope activity by using a scintillation counter are more expensive and have the issue concerning the safety of isotopes, as compared to the methods used before.

In the prior art, there are methods for directly determining the MVAL content by LC-MS/MS. However, the methods cannot accurately reflect the entire amount of the MVA produced in the system. That is because HMG-CoA produces MVA by using HMG-CoA reductase in an enzyme reaction system, and when acid is added to terminate the reaction, the MVA thus produced is converted to MVAL but it cannot be ensured that the MVA is completely converted to MVAL.

Xuezhikang capsule, a medicine for treating hyperlipoidemia that comprises red yeast rice as the main ingredient, contains a variety of HMG-CoA reductase inhibitor components (Ma J, Li Y, Ye Q, et al. Constituents of Red Yeast Rice, a Traditional Chinese food and medicine. J Agric Food Chem. 2000, 48:5220). However, since many of the components are not known, it is difficult to determine the contents of the HMG-CoA reductase inhibitors in Xuezhikang capsule.

Therefore, methods for accurately determining the inhibition rate of HMG-CoA reductase and methods for determining the concentration of HMG-CoA reductase inhibitors in an unknown sample are urgent in the field.

### CONTENTS OF INVENTION

After conducting deep research and paying creative work, the inventors surprisingly found a method for determining the inhibition rate of HMG-CoA reductase, and by combining the method, the concentration or content of HMG-CoA reductase inhibitors can be effectively and accurately determined in a sample containing unknown specific components (such as Xuezhikang capsule). Therefore, the invention is provided as follows.

In one aspect, the invention relates to a method for determining the inhibition rate of HMG-CoA reductase, which comprises the following steps:

1) establishing the following enzymatic reaction systems for HMG-CoA reductase:

a reaction system for a sample to be tested, to which a sample to be tested is added, and

a negative control reaction system, to which deionized water in the same volume as a sample to be tested is added;

2) determining the MVAL(Mevalonolactone) concentration in a reaction system for a sample to be tested and in a negative control reaction system by HPLC-MS/MS method, respectively;

3) calculating the inhibition rate of HMG-CoA reductase according to the formula:

inhibition rate of HMG-CoA reductase=[(MVAL concentration in a negative control reaction system−MVAL concentration in a reaction system for a sample to be tested)/MVAL concentration in a negative control reaction system]×100%.

MVA is produced from HMG-CoA by using HMG-CoA reductase in an enzyme reaction system, and the MVA thus produced is converted to MVAL when acid is added to terminate the reaction, but it cannot be ensured that the MVA is completely converted to MVAL. In the invention, MVA is completely converted to MVAL (which may be accomplished by the addition of acid such as hydrochloric acid; the acid may also serve to terminate the enzymatic reaction); the MVAL completely converted to MVA (for example, which may be accomplished by addition of alkali such as aqueous ammonia); and then the concentration of the resultant MVA is determined. Namely, MVA undergoes a conversion of MVA→MVAL→MVA. In one embodiment of the invention, the MVAL concentration is obtained by the following steps:

a. completely converting MVA to MVAL in each of the reaction systems;

b. completely converting the MVAL in step a to MVA;

c. determining the MVA concentration in step b; and

d. using the MVA concentration determined in step c as the MVAL concentration.

In another aspect, MVAL is generally taken as a marker for enzyme action, and therefore the determined MVA concentration is represented as the MVAL concentration. Thus, in the invention, the MVAL concentration in a reaction system for a sample to be tested may also be used as the MVA concentration in the reaction system for a sample to be tested; and the MVAL concentration in a negative control reaction system may also be used as the MVA concentration in the negative control reaction system.

In the method for determining the inhibition rate of HMG-CoA reductase according to any item of the invention, the sample to be tested is a HMG-CoA reductase inhibitor, such as lovastatin hydroxy acid. The sample to be tested may be in a form of liquid or solid. If it is in a form of solid, a solution may be prepared from it and be used as a sample to be tested.

In one embodiment of the invention, in step 1), establishment of enzymatic reaction systems for HMG-CoA reductase, comprises the following steps:

pipetting 100 μL blank human plasma (i.e. pure plasma obtained by adding no additive to human plasma) into a 1.2 mL deep well in a 96-well plate, and mixing the resultant solution uniformly under vortexing after adding 400 μL methanol, centrifugating the sample solution at 3750 rpm for 10 min, pipetting 300 μL supernatant to a 1.2 mL tube in a dismountable 96-well plate, and drying the supernatant at 40° C. under nitrogen blowing;

adding 20 μL sample to be tested with a pipette to the tube to obtain the reaction system for a sample to be tested (adding no sample to be tested to the tube to obtain the negative control reaction system);

placing the reaction system in an ice bath, then adding 120 μL solution of rat liver microsome (3.3 mg/ml) (purchased from Research Institute for Liver Diseases (Shanghai) Co., Ltd), and vortexing the mixture for 5 s;

incubating the mixture at 37° C. in a water bath under shaking for 15 min; further adding 20 μL HMG-CoA solution (0.5 mM), and mixing the resultant solution uniformly under vortexing;

incubating the mixture at 37° C. in a water bath under shaking for 30 min;

adding 20 μL HCl (5N), mixing the resultant solution uniformly under vortexing;

incubating the mixture at 37° C. in a water bath under shaking for 15 min to terminate the reaction, wherein the reaction solution may be used for the determination of the MVAL concentration.

In the method for determining the inhibition rate of HMG-CoA reductase according to any item of the invention, in step 2), determination of the MVAL concentration in a negative control reaction system and in a reaction system for a sample to be tested by HPLC-MS/MS method, respectively, comprises the following steps:

A. adding hydrochloric acid to the negative control reaction system and the reaction system for a sample to be tested, respectively, mixing and standing to convert MVA (Mevalonic acid) to MVAL (Mevalonolactone);

B. pre-treating an ENV-SPE small column with methanol and 0.1N hydrochloric acid successively;

C. loading the sample solutions obtained in step A to the ENV-SPE small column, respectively;

D. eluting the ENV-SPE small column with 0.1N hydrochloric acid and deionized water successively;

E. eluting with methanol the ENV-SPE small column treated in step D, enriching the fraction and obtaining an eluate;

F. drying the eluate obtained in step E to obtain a dried product;

redissolving the dried product obtained in step F with aqueous ammonia, mixing and standing to convert MVAL to MVA; and

H. injecting the sample obtained in step G into a HPLC-MS/MS system.

In the method for determining the inhibition rate of HMG-CoA reductase according to any item of the invention, the drying in step F is carried out at 40° C. under nitrogen blowing.

In the method for determining the inhibition rate of HMG-CoA reductase according to any item of the invention, the aqueous ammonia in step G has a concentration of 0.2%.

In the method for determining the inhibition rate of HMG-CoA reductase according to any item of the invention, the standing in step A and/or G is carried out for 30 minutes.

In the method for determining the inhibition rate of HMG-CoA reductase according to any item of the invention, in step H, the sample is stabilized at 15° C. in an automatic sample injector for 24 hours.

In the method for determining the inhibition rate of HMG-CoA reductase according to any item of the invention, the HPLC conditions are as follows:

| | | |
|---|---|---|
| Mobile phase | 10 | mM ammonium formate (pH 8.0): |
| | | acetonitrile, 70/30 (v/v) |
| Flow rate | 0.8 | mL/min (splitless) |
| Solution for washing needle | 50:50 | methanol/water (v/v) |
| Injection volume | 30 | μL |
| Time for data acquisition | 3 | min |

| | |
|---|---|
| Column temperature | room temperature |
| Autosampler temperature | room temperature (such as 15° C.); |

the switch time T1 of the switching valve is set at least 0.5 minutes before the starting time of the chromatographic peak of interest, and the switch time T2 is set at least 0.5 minutes after the ending time of the chromatographic peak of interest;

MS/MS Conditions:

MVA:

| Polarity | Negative mode | |
|---|---|---|
| Mass-to-charge ratio (m/z) of parent ion | 147.0 | |
| Mass-to-charge ratio (m/z) of daughter ion | 59.1 | |
| Dwell time | 200 | msec |
| Pause time | 5 | msec |
| Retention time | about 1.8 | min; |

MVA-d₇:

| Polarity | Negative mode | |
|---|---|---|
| Parent ion m/z | 154.0 | |
| Daughter ion m/z | 59.1 | |
| Dwell time | 200 | msec |
| Pause time | 5 | msec |
| Retention time | about 1.8 | min |

In one embodiment of the invention, the switch time T1 is 1.2 min, and the switch time T2 is 2.5 min.

The above-mentioned HPLC-MS/MS may be commercially available High Performance Liquid Chromatography-Mass Spectrometer (HPLC-MS), for example, API 4000™ LC/MS/MS from US Applied Biosystems.

In the method for determining the inhibition rate of HMG-CoA reductase according to any item of the invention, the MVAL concentration is calculated as follows:

determining the retention times and peak areas of chromatographic peaks, establishing a curve according to the peak area ratios and the concentrations, and calculating the MVAL concentration according to the curve, i.e. calculating the MVAL concentration by linear regression according to the following formula:

y=ax+b
wherein

y=peak area ratio of an test substance (MVA) and an internal standard (MVA-d₇),

b=intercept of the curve,

a=slope of the curve,

x=MVAL concentration (the actually determined concentration is the MVA concentration, since MVAL is generally taken as a marker for enzyme action, the determined MVA concentration is represented as the MVAL concentration).

In particular, the retention times and peak areas of chromatographic peaks may be determined by analysis softwares (which may be analysis softwares known in the art, for example, softwares built in HPLC, such as Applied Biosystems Analyst Data Acquisition Software(version 1.4.1)), and a curve is established according to the peak area ratios and the concentrations.

In the method for determining the inhibition rate of HMG-CoA reductase according to any item of the invention, the MVAL concentration in the negative control reaction system refers to an average concentration in several negative control reaction systems.

In a preferred embodiment of the present invention, the MVA content is finally determined by a MVA→MVAL→MVA method. Since MVAL is generally taken as a marker for enzyme action, the determined MVA concentration is represented as the MVAL concentration. The MVAL concentration is calculated by the formula y=ax+b.

In another aspect, the invention relates to a method for determining the concentration of a HMG-CoA reductase inhibitor in a sample to be tested, which comprises the following steps:

I) determining the inhibition rates of HMG-CoA reductase corresponding to n groups of lovastatin hydroxy acid solutions with known concentrations by the method according to any of the preceding items, n≧5, preferably, n≧10;

II) establishing a curve equation of the inhibition rate y of HMG-CoA reductase—the concentration X of lovastatin hydroxy acid solution in step 1);

III) determining the inhibition rate y of HMG-CoA reductase in a sample to be tested, by the method according to any of the preceding items,

IV) calculating the concentration X of lovastatin hydroxy acid solution, i.e. the concentration of the HMG-CoA reductase inhibitor in the sample to be tested, by applying the inhibition rate y of HMG-CoA reductase in a sample to be tested, as obtained in step III), to the curve equation in step II).

In one embodiment of the invention, the curve equation is established by software Origin 7.5 in step II).

In one embodiment of the invention, the curve equation in step II) is as follows:

a curve equation of the inhibition rate y of HMG-CoA reductase—the concentration X of lovastatin hydroxy acid:

y=A₂+(A₁−A₂)/[1+(X/X₀)^{P}]

wherein

X is the concentration of lovastatin hydroxy acid or the concentration of an inhibitor (ng/mL),

−5≦A₁<A₂≦115,

A₁, A₂, X₀ and P are original parameters from software Origin 7.5. Said four parameters are parameters for curve fitting, which decide the shape of the curve, wherein A₁ is the top of the fitted curve, the valuation of upper asymptote; A₂ is the bottom of the fitted curve, the valuation of lower asymptote; X₀ is the Midpoint of the fitted curve, a median; and P is slope, hillslope. The four parameters are little changed in different batches of reactions.

In one embodiment of the invention, the sample is a Xuezhikang capsule or a solution prepared from a Xuezhikang capsule. A person skilled in the art can readily calculate the content of HMG-CoA reductase inhibitor in each Xuezhikang capsule, according to the determined concentration of HMG-CoA reductase inhibitor in a solution prepared from Xuezhikang capsule, in combination with the volume of the solution and the number and/or weight of the Xuezhikang capsule used.

### ADVANTAGEOUS EFFECTS OF THE INVENTION

The research and development of statins have been one of the research hotspots in the field of natural products and microbes. Assays in vitro are essential for the research and development of statins, and in both research and large-scale production, control of product quality, as well as quantitative and quantitative determination of compounds are very important.

The interference to spectrophotography, as caused by the insoluble components from drugs (e.g. Xuezhikang capsule), can be eliminated when evaluating HMG-CoA reductase inhibitory activity in natural drugs (such as Xuezhikang capsule) comprising a variety of components with HMG-CoA reductase inhibitory activity, by utilizing an enzymatic reaction system in vitro and LC-MS/MS system. It can accurately reflect the enzyme inhibitory activity of the drugs, and determine the total content of the active components in one step without determining the content of each of the components one by one. The activity and the content of any new possible HMG-CoA reductase inhibitor can be confirmed by the method. The method for determining the concentration of an HMG-CoA reductase inhibitor in a sample to be tested, according to the invention, has a high sensitivity and a good accuracy.

### DESCRIPTION OF DRAWINGS

FIG. 1: the calibration curve of MVAL.

FIG. 2: the standard curve of lovastatin hydroxy acid.

FIG. 3: FIG. 3 (A) depicts a chromatogram for a Xuezhikang capsule sample; FIG. 3 (B) depicts a chromatogram for an internal standard of MVA, i.e. MVA-d₇. Note: the “peak name” in FIG. 3 (A) actually refers to MVA, since MVAL is generally taken as a marker for enzyme action, it is represented as MVAL.

### SPECIFIC MODE FOR CARRYING OUT THE INVENTION

The embodiments of the invention are described in detail by combining the following Examples. However, a person skilled in the art would understand that the following Examples are intended to illustrate the invention, rather than limiting the scope of the invention. Under the circumstance where the specific techniques or conditions are not indicated in the Examples, the embodiments are carried out according to the techniques or conditions described in the literature of the art or according to the specifications of the products. The reagents or equipments, the manufacturers of which are not indicated, are routine products that are commercially available.

### Example 1

### Determination of the Inhibition Rate of HMG-CoA Reductase

1. Establishment of an enzymatic reaction system for HMG-CoA reductase 100 μL blank human plasma (which provided a suitable reaction environment for the enzymatic reaction) was pipetted into a 1.2 mL deep well in a 96-well plate. After adding 400 μL methanol, the resultant solution was mixed uniformly under vortexing; the sample solution was centrifugated at 3750 rpm for 10 min, 300 μL supernatant was pipetted into a 1.2 mL tube in a dismountable 96-well plate, and the supernatant was dried at 40° C. under nitrogen blowing.

20 μL sample (the sample to be tested was a lovastatin hydroxy acid solution with a concentration shown in Table 1, and the sample of the negative control was deionized water) was added into each tube with a pipette.

In an ice bath, 120 μL solution of rat liver microsome (3.3 mg/ml) was pipetted into each tube with a multi-channel pipette, and the mixture was vortexed for 5 s.

The mixture was incubated in a water bath at 37° C. under shaking for 15 min.

20 μL HMG-CoA solution (0.5 mM) was added with a multi-channel pipette, and the resultant solution was mixed uniformly under vortexing.

The resultant solution was incubated in a water bath at 37° C. under shaking for 30 min; 20 μL, HCl (5N) was pipetted into each well with a multi-channel pipette, and the resultant solution was mixed uniformly under vortexing. The resultant solution was incubated in a water bath at 37° C. under shaking for 30 min to terminate the reaction. The reaction solution might be used for the determination of the MVAL concentration.

Standards (samples to be tested) were lovastatin hydroxy acid solutions, the volume for each of them was 1000 μL, and their concentrations were shown in Table 1.

TABLE 1
10 lovastatin hydroxy acid solution samples
as standards and the concentrations thereof

| | Concentration |
|---|---|
| | (lovastatin hydroxy acid) |
| Sample Name | (Lov acid) (ng/mL) |
| STD 1 | 1.25 |
| STD 2 | 2.5 |
| STD 3 | 5 |
| STD 4 | 10 |
| STD 5 | 25 |
| STD 6 | 50 |
| STD 7 | 75 |
| STD 8 | 100 |
| STD 9 | 125 |
| STD 10 | 150 |

2. Determination of the MVAL concentration by HPLC-MS/MS method

HPLC Conditions:

| | | |
|---|---|---|
| Mobile phase | 10 | mM ammonium formate (pH 8.0): |
| | | acetonitrile, 70/30 (v/v) |
| Flow rate | 0.8 | mL/min (splitless) |
| Solution for washing the | 50:50 | methanol/water (v/v) |
| needle | | |
| Injection volume | 30 | μL |
| Time for data acquisition | 3 | min |

| | |
|---|---|
| Column temperature | room temperature |
| Autosampler temperature | room temperature (such as 15° C. ); |

Switching Valve Conditions:

Switch time: T1 was about 1.2 min, T2 was about 2.5 min.

Note: The specific switch time and data acquisition time vary depending on the chromatographic column and chromatographic conditions. The switch time T1 of the switching valve should be set at least 0.5 minutes before the starting time of the chromatographic peak of interest, and the switch time T2 should be set at least 0.5 minutes after the ending time of the chromatographic peak of interest.

MS/MS Conditions:

Mevalonic Acid (MVA):

| Polarity | Negative mode | |
|---|---|---|
| Mass-to-charge ratio (m/z) of parent ion | 147.0 | |
| Mass-to-charge ratio (m/z) of daughter ion | 59.1 | |
| Dwell time | 200 | msec |
| Pause time | 5 | msec |
| Retention time | about 1.8 | min; |

MVA-d₇ (an Internal Standard of MVA, MVA-d₇ is Deuterium-Substituted MVA):

| Polarity | Negative mode | |
|---|---|---|
| Parent ion m/z | 154.0 | |
| Daughter ion m/z | 59.1 | |
| Dwell time | 200 | msec |
| Pause time | 5 | msec |
| Retention time | about 1.8 | min; |

The internal standard solution was prepared as followed.

A stock solution (ISS, 500 μg/mL) of an internal standard (MVAL-d₇, i.e. deuterium-substituted MVAL): 2.5 mg MVAL-d₇ was added into a 5 mL volumetric flask, dissolved with acetonitrile and diluted to a final volume of 5 mL. The solution was stored at 4° C. (The solution was stabilized at −20° C. for 82d).

An internal standard spike solution (IS Spike 200 ng/mL): 20 μL ISS internal standard stock solution was pipetted into a 50 mL volumetric flask, dissolved with purified water and diluted to a final volume of 50 mL, and then shaked up. The solution was stored at 4° C. (The solution was stabilized at 4° C. for 22d).

The Preparation of MVAL Samples.

150 μL MVAL standard solution or other reaction solutions (double-blank solution, blank solution, standard solution, sample solution to be tested) were added to a glass tube.

Except for the double-blank solution, 100 μL internal standard spike solution was added to each of the rest tubes.

900 μL 0.1N HCl and 1 mL water was added, and the resultant mixtures were mixed uniformly under vortexing, and standed for 30 min to convert MVA to MVAL.

An ENV-SPE small column (purchased from Supelco Inc, US.) was pre-treated with 1 mL methanol and 1 mL 0.1N HCl successively.

0.5 mL sample solution was loaded to the ENV-SPE small column.

After loading the sample, the small column was washed with 1 mL 0.1N HCl and 1 mL purified water successively.

The sample was eluted with 5*0.5 mL methanol, and the fraction was enriched.

The eluate was dried at 40° C. under nitrogen blowing.

The dried product was redissolved with 200 μL 0.2% aqueous ammonia, and was mixed uniformly under vortexing, and then standed for 30 min to convert MVAL to MVA.

30 μL sample was injected into a LC/MS/MS system. The sample solution was stabilized at 15° C. in an automatic sample injector for 24 hours.

Quantitative Analysis of MVAL.

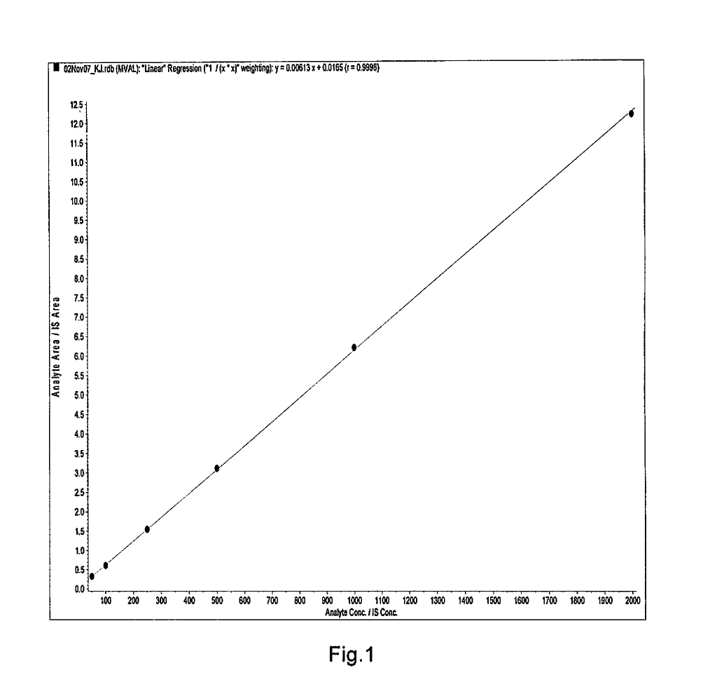

The retention times and peak areas of chromatographic peaks may be determined by analysis softwares (softwares built in HPLC, such as Applied Biosystems Analyst Data Acquisition Software (version 1.4.1)). A calibration curve (as shown in FIG. 1) was established according to the peak area ratios and the concentrations, and the MVAL concentration may be calculated according to the curve.

The MVAL concentration may be calculated by linear regression according to the following formula:

y=ax+b

wherein

y=ratio of the peak area of a test substance (MVA) and the peak area of an internal standard (MVA-d₇),

b=intercept of the curve,

a=slope of the curve,

x=concentration (ng/mL) of a test substance (MVAL) (regression calculation is carried out by weighted least square method).

In the reaction systems of 10 lovastatin hydroxy acid solutions (STD1-STD10), the MVAL concentrations as calculated were 723.085, 728.387, 634.108, 553.920, 425.730, 324.551, 269.938, 194.026, 180.493 and 155.692 ng/mL, respectively.

In 4 negative control reaction systems, the MVAL concentrations as calculated were 857.248, 819.397, 841.799 and 806.182 ng/mL, respectively.

Note: Since no sample solution was added to a negative control system and only purified water in the same volume as the sample solution was added, one negative control was enough theoretically. For insurance, four negative controls were employed. No matter one or four negative controls were used, the effects were the same.

3. Calculation of the inhibition rate of HMG-CoA reductase.

The inhibition rate of HMG-CoA reductase was calculated according to the formula:

inhibition rate of HMG-CoA reductase=[(MVAL concentration in a negative control reaction system−MVAL concentration in a reaction system for a sample to be tested)/MVAL concentration in a negative control reaction system]×100%.

By applying the MVAL concentrations in the negative control reaction system and the MVAL concentrations in reaction systems for the samples to be tested (lovastatin hydroxy acid standard solutions STD1-STD10) to the above formula, the inhibition rates of HMG-CoA reductase in STD1-STD10 were calculated, which were 13.00, 12.36, 23.71, 33.36, 48.78, 60.95, 67.52, 76.66, 78.28, and 81.27%, respectively.

### Example 2

### Drawing of the Standard Curve of the Inhibition Rate of HMG-CoA Reductase—Lovastatin Hydroxy Acid Concentration

On the basis of the inhibition rates of HMG-CoA reductase in STD1-STD10 obtained in Example 1, and the corresponding lovastatin hydroxy acid concentrations of the standards STD 1-STD 10 in Table 1, the logarithmic dose response method in pharmacological/chemical software Origin 7.5 was employed.

y=A₂+(A₁−A₂)/[1+(X/X₀)^{P}]

wherein

X=the concentration of lovastatin hydroxy acid (ng/mL) (without weighted processing)

−5≦A₁<A₂≦115

A₁, A₂, X₀ and P were original parameters from software Origin 7.5. During interaction process, simplified chi-square test was not reduced.

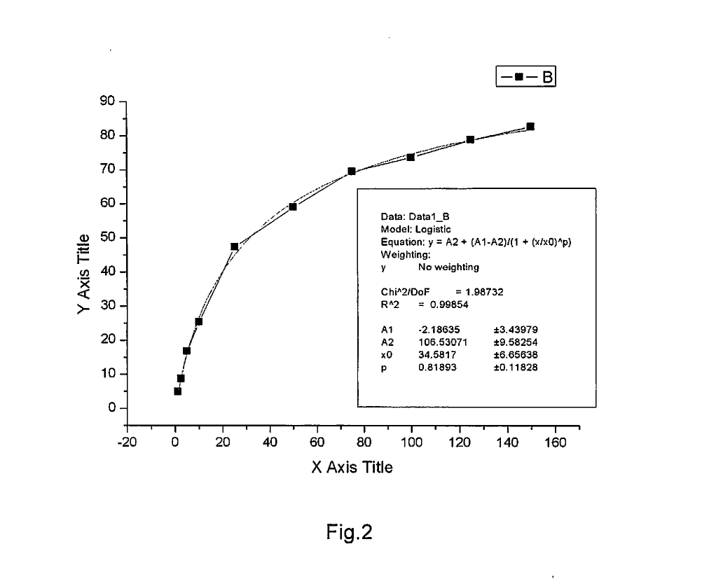

The obtained standard curve was shown in FIG. 2.

### Example 3

### Calculation of the Concentrations of HMG-CoA Reductase Inhibitors in a Solution of Xuezhikang Capsule

The inhibition rate of HMG-CoA reductase in a solution of Xuezhikang capsule was obtained by the method of Example 1, except that a solution of Xuezhikang capsule was used in place of a sample to be tested. A solution of Xuezhikang capsule was prepared as followed.

6 Xuezhikang capsules (6 parallel samples) were opened, and the contents were added to suitable containers, weighted and recorded. The contents contained in the capsules were pulverized and mixed uniformly. About 0.3 g powder content weighted precisely for six times was placed in a 25 mL volumetric flask, and about 20 mL diluent was added. After ultrasonic treatment for 10 min, dilutent was added to a final volume of 25 mL. The diluent was pipetted into a PTEE centrifuge tube and was contrifugated at 3750 rpm for 5 min. 75 μL supernatant was pipetted into a 100 mL volumetric flask, and water was added to a final volume of 100 mL. The sample solution was stabilized at 4° C. for 24 h.

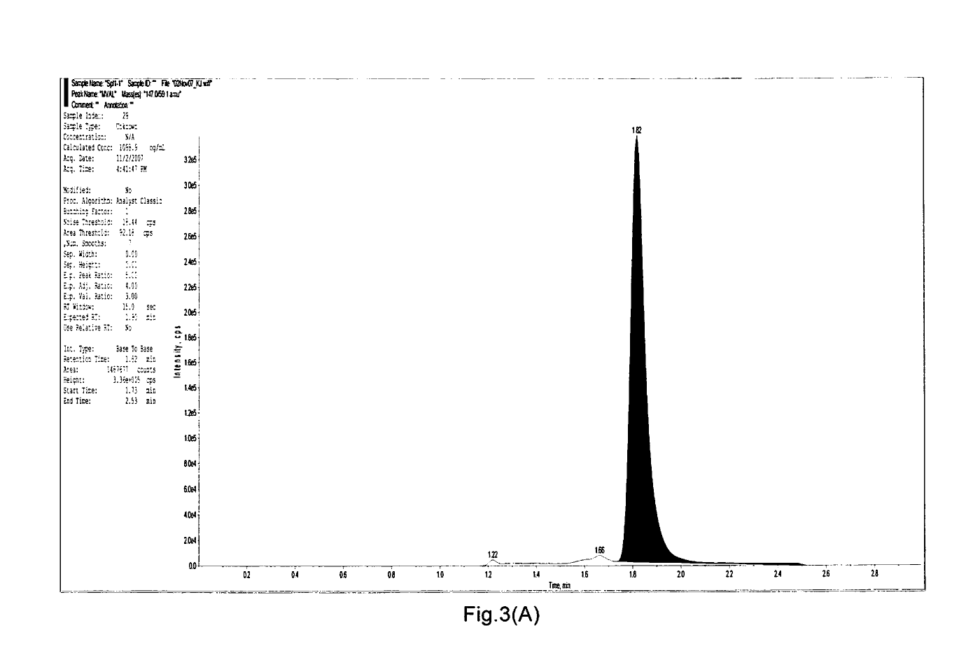

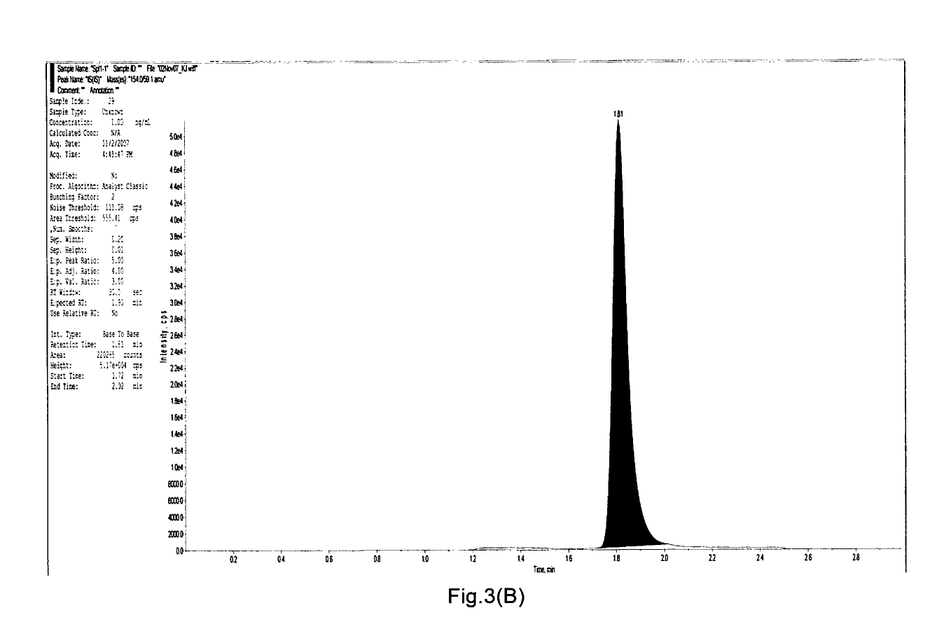

The chromatograph of the solution sample of Xuezhikang Capsule was shown in FIG. 3A (FIG. 3B showed the chromatograph of an internal standard of MVA, i.e. MVA-d7).

According to the method of Example 1, the inhibition rate of HMG-CoA reductase in a solution of Xuezhikang capsule was obtained, i.e. the average inhibition rates of the 6 samples were 47.94%, 46.14%, 43.75%, 47.13%, 45.69% and 45.02%, respectively.

By applying the inhibition rates to the formula of Example 2, the concentration of the HMG-CoA reductase inhibitor in a solution of Xuezhikang capsule was calculated. The concentrations of HMG-CoA reductase inhibitor in the 6 samples were 24.842, 22.895, 20.051, 23.991, 21.929 and 21.623 ng/mL, respectively.

### Example 4

### Calculation of the Content of HMG-CoA Reductase Inhibitor in a Xuezhikang Capsule

According to the concentrations and volumes of the capsule solutions of Example 3, as well as the weights of the 6 Xuezhikang capsule samples (300.07, 300.83, 299.48, 299.67, 300.78, and 300.02 mg, respectively), the content of HMG-CoA reductase inhibitor in each capsule sample was calculated. The contents of HMG-CoA reductase inhibitor in the 6 samples were 0.446, 0.410, 0.361, 0.431, 0.393, and 0.388 mg/capsule, respectively.

### Example 5

### Proof Test of Sensitivity and Accuracy (1)

The lower limit of lovastatin hydroxy acid to be quantified was set to be 5 ng/mL. In order to assess sensitivity and accuracy of the method, 6 samples were tested at the concentration. The test method was similar to the one in Example 3, except that lovastatin hydroxy acid with a known concentration was used in place of a sample to be tested. The results were shown in Table 2. For a sample comprising a lower limit of lovastatin hydroxy acid to be quantified, the concentration as calculated had an accuracy of 84.2%.

TABLE 2
Tests on sensitivity and accuracy of low concentrations of samples

| | | lovastatin | | | |
|---|---|---|---|---|---|
| | lovastatin | hydroxy acid | | | |
| | hydroxy acid | concentration | | | Relative |
| | concen- | (ng/mL) obtained | | | standard |
| Sample | tration | by retroactive | Average | Accu- | deviation |
| No. | (ng/mL) | calculation | value | racy | RSD % |
| 1 | 5 | 4.173 | 4.208 | 84.2% | 10.5 |
| 2 | 5 | 4.487 | | | |
| 3 | 5 | 3.619 | | | |
| 4 | 5 | 4.421 | | | |
| 5 | 5 | 3.773 | | | |
| 6 | 5 | 4.775 | | | |

It can be seen from Table 2 that the method for determining the concentration of HMG-CoA reductase inhibitor according to the invention has a high sensitivity and a good accuracy.

### Example 6

### Proof Test of Sensitivity and Accuracy (2)

By reference to a method similar to the one in Example 5, the concentrations of standard lovastatin hydroxy acid solutions with known concentrations were determined. The samples used and the results were shown in Table 3.

TABLE 3
Tests on sensitivity and accuracy of
samples at gradient concentrations

| | | lovastatin hydroxy | |
|---|---|---|---|
| | lovastatin | acid concentration | |
| | hydroxy acid | (ng/mL) obtained | |
| Sample | concentration | by retroactive | Accuracy |
| No. | (ng/mL) | calculation | (%) |
| 1 | 5 | 5.344 | 106.9 |
| 2 | 10 | 10.659 | 106.6 |
| 3 | 25 | 25.702 | 102.8 |
| 4 | 50 | 48.264 | 96.5 |
| 5 | 75 | 67.995 | 90.7 |
| 6 | 100 | 112.540 | 112.5 |
| 7 | 125 | 123.823 | 99.1 |

It can be seen from Table 3 that the method for determining the concentration of HMG-CoA reductase inhibitor according to the invention has a high sensitivity and a good accuracy.

Although the specific embodiments of the invention have been described in detail, a person skilled in the art would understand that according to all the teachings as disclosed, various modification and substitutions may be made to the details, and these changes fall into the protection scope of the invention. The full scope of the invention is defined by the appended claims and any other equivalent.

What we claim is:

1. A method for determining the inhibition rate of HMG-CoA reductase, which comprises the following steps:
1) establishing the following enzymatic reaction systems for HMG-CoA reductase:
a reaction system for a sample to be tested, to which a sample to be tested is added, and
a negative control reaction system, to which deionized water in the same volume as the sample to be tested is added;
2) determining the MVAL concentration in a negative control reaction system and in a reaction system for a sample to be tested by HPLC-MS/MS method, respectively; and
3) calculating the inhibition rate of HMG-CoA reductase according to the formula:
inhibition rate of HMG-CoA reductase=[(MVAL concentration in a negative control reaction system−MVAL concentration in a reaction system for a sample to be tested)/MVAL concentration in a negative control reaction system]×100%.

2. The method according to claim 1, wherein the MVAL concentration is obtained by the following steps:
a. completely converting MVA to MVAL in each of the reaction systems;
b. completely converting the MVAL in step a to MVA;
c. determining the MVA concentration in step b; and
d. using the MVA concentration determined in step c as the MVAL concentration.

3. The method according to claim 1, wherein the sample to be tested is a HMG-CoA reductase inhibitor, such as lovastatin hydroxy acid.

4. The method according to claim 1, wherein in the step 2), determination of the MVAL concentration in a negative control reaction system and in a reaction system for a sample to be tested by HPLC-MS/MS method, respectively, comprises the following steps:
A. adding hydrochloric acid to the negative control reaction system and the reaction system for a sample to be tested, respectively, mixing and standing, to convert MVA to MVAL;
B. pre-treating an ENV-SPE small column with methanol and 0.1N hydrochloric acid successively;
C. loading the sample solutions obtained in step A to the ENV-SPE small column, respectively;
D. eluting the ENV-SPE small column with 0.1N hydrochloric acid and deionized water successively;
E. eluting with methanol the ENV-SPE small column treated in step D, enriching the fraction and obtaining an eluate;
F. drying the eluate obtained in step E to obtain a dried product;
G. redissolving the dried product obtained in step F with aqueous ammonia, mixing and standing, to convert MVAL to MVA; and
H. injecting the sample obtained in step G into a HPLC-MS/MS system.

5. The method according to claim 4, wherein it satisfies one or more of the following items (1)-(5):
(1) the standing in step A is carried out for 30 minutes;
(2) the drying in step F is carried out at 40° C. under nitrogen blowing;
(3) the aqueous ammonia in step G has a concentration of 0.2%;
(4) the standing in step G is carried out for 30 minutes; and
(5) in step H, the sample is stabilized at 15° C. in an automatic sample injector for 24 hours.

6. The method according to claim 4, wherein the HPLC conditions are as follows:

| Mobile phase | 10 | mM ammonium formate (pH 8.0): |
|---|---|---|
| | | acetonitrile, 70/30 (v/v) |
| Flow rate | 0.8 | mL/min (splitless) |
| Solution for washing needle | 50:50 | methanol/water (v/v) |
| Injection volume | 30 | μL |
| Time for data acquisition | 3 | min |
| Column temperature | room temperature | |
| Autosampler temperature | 15° | C.; |

the switch time T1 of the switching valve is set at least 0.5 minutes before the starting time of the chromatographic peak of interest, and the switch time T2 is set at least 0.5 minutes after the ending time of the chromatographic peak of interest;
MS/MS conditions:
MVA:

| Polarity | Negative mode | |
|---|---|---|
| Mass-to-charge ratio (m/z) of parent ion | 147.0 | |
| Mass-to-charge ratio (m/z) of daughter ion | 59.1 | |
| Dwell time | 200 | msec |
| Pause time | 5 | msec |
| Retention time | about 1.8 | min; |

MVA-d₇:

| Polarity | Negative mode | |
|---|---|---|
| Parent ion m/z | 154.0 | |
| Daughter ion m/z | 59.1 | |
| Dwell time | 200 | msec |
| Pause time | 5 | msec |
| Retention time | about 1.8 | min. |

7. The method according to claim 6, wherein the MVAL concentration is calculated as follows:
determining the retention times and peak areas of chromatographic peaks, establishing a curve according to the peak area ratios and the concentrations, and calculating the MVAL concentration according to the curve, i.e. calculating the MVAL concentration by linear regression according to the following formula:
y=ax+b
wherein
y=peak area ratio of MVA and an internal standard MVA-d₇,
b=intercept of the curve,
a=slope of the curve,
x=MVAL concentration.

8. A method for determining the concentration of a HMG-CoA reductase inhibitor in a sample to be tested, comprising the following steps:
I) determining the inhibition rates of HMG-CoA reductase corresponding to n groups of lovastatin hydroxy acid solutions with known concentrations by the method according to any one of claim 1-7, n≧5;
II) establishing a curve equation of the inhibition rate y of HMG-CoA reductase—the concentration X of lovastatin hydroxy acid solution in step I);
III) determining the inhibition rate y of HMG-CoA reductase in a sample to be tested, by the method according to any one of claim 1-7, and
IV) calculating the concentration X of lovastatin hydroxy acid solution, i.e. the concentration of the HMG-CoA reductase inhibitor in the sample to be tested, by applying the inhibition rate y of HMG-CoA reductase in a sample to be tested, as obtained in step III), to the curve equation in step II).

9. The method according to claim 8, wherein the curve equation is established by software Origin 7.5 in step II).

10. The method according to claim 8, wherein the curve equation in step II) is as follows:
a curve equation of the inhibition rate y of HMG-CoA reductase—the concentration X of lovastatin hydroxy acid
y=A₂+(A₁−A₂)/[1+(X/X₀)^{P}]
wherein
X is the concentration of lovastatin hydroxy acid or the concentration of an inhibitor (ng/mL),
−5≦A₁<A₂≦115,
A₁, A₂, X₀ and P are original parameters from software Origin 7.5.

11. The method according to claim 2, wherein the sample to be tested is a HMG-CoA reductase inhibitor, such as lovastatin hydroxy acid.

12. The method according to claim 2, wherein in the step 2), determination of the MVAL concentration in a negative control reaction system and in a reaction system for a sample to be tested by HPLC-MS/MS method, respectively, comprises the following steps:
A. adding hydrochloric acid to the negative control reaction system and the reaction system for a sample to be tested, respectively, mixing and standing, to convert MVA to MVAL;
B. pre-treating an ENV-SPE small column with methanol and 0.1N hydrochloric acid successively;
C. loading the sample solutions obtained in step A to the ENV-SPE small column, respectively;
D. eluting the ENV-SPE small column with 0.1N hydrochloric acid and deionized water successively;
E. eluting with methanol the ENV-SPE small column treated in step D, enriching the fraction and obtaining an eluate;
F. drying the eluate obtained in step E to obtain a dried product;
G. redissolving the dried product obtained in step F with aqueous ammonia, mixing and standing, to convert MVAL to MVA; and
H. injecting the sample obtained in step G into a HPLC-MS/MS system.
